(19) United States
(12) Patent Application Publication
(10) Pub. No.: US 2024/0191169 A1
(43) Pub. Date: Jun. 13, 2024

(54) CELL CULTURE APPARATUS AND CELL CULTURE METHOD

(71) Applicants: Canon Medical Systems Corporation, Otawara-shi (JP); CANON KABUSHIKI KAISHA, Tokyo (JP)

(72) Inventor: Katsuaki DEGUCHI, Otawara (JP)

(73) Assignees: Canon Medical Systems Corporation, Otawara-shi (JP); CANON KABUSHIKI KAISHA, Tokyo (JP)

(21) Appl. No.: 18/531,789

(22) Filed: Dec. 7, 2023

(30) Foreign Application Priority Data
Dec. 8, 2022 (JP) 2022-196501

(51) Int. Cl. C12M 1/12; C12M 1/26; C12M 3/04

(52) U.S. Cl. CPC C12M 25/10; C12M 27/10; C12M 33/12

(57) ABSTRACT

According to one embodiment, a cell culture apparatus includes a culture container, a motor, an ejecting pipe, and a discharging pipe. The culture container has a transverse hollow tubular shape, has an opening formed in the center of a bottom surface, and has an openable and closable lid portion formed on a side surface. The motor rotates the culture container around a rotation axis perpendicular to the bottom surface. The ejecting pipe is inserted from the opening into the culture container. The discharging pipe is connected to the lid portion. An inner wall surface of the culture container has a shape of a single spiral when viewed from the direction of the rotation axis. The ejecting pipe ejects a liquid onto the inner wall surface. The discharging pipe discharges the ejected liquid from the lid portion.

### CROSS-REFERENCE TO RELATED APPLICATION (S)

This application is based upon and claims the benefit of priority from Japanese Patent Application No. 2022-196501, filed Dec. 8, 2022, the entire contents of which are incorporated herein by reference.

### FIELD

Embodiments described herein relate generally to a cell culture apparatus and a cell culture method.

### BACKGROUND

Conventionally, there have been many closed-system cell culture apparatuses. However, it is not easy for closed-system cell culture apparatuses to perform processes relating to cell cultures (such as seeding, culturing, peeling, concentration, and collection) in a single culture container.

In the peeling process, for example, a cell culture apparatus needs to physically peel cells from a medium by ejecting a solution onto the cells on the medium with a predetermined pressure. On the other hand, in the concentration process, a cell culture apparatus concentrates a cell suspension using a filter or centrifugation. However, using a filter results in losing many cells. In the case of centrifugation, a cell culture apparatus needs to transfer a cell suspension in a culture container to a different container to perform centrifugation, then remove a supernatant after the centrifugation, and re-suspend the cells in a different solution. Thus, in the case of centrifugation, the structure of the cell culture apparatus tends to be complicated.

### BRIEF DESCRIPTION OF THE DRAWINGS

FIG. 1 is a schematic diagram showing an example of a configuration of a cell culture apparatus according to an embodiment.

FIG. 2 is a schematic diagram showing an example of a configuration of a culture container of the cell culture apparatus according to the embodiment.

FIG. 3 is a flowchart of a cell culture process performed by the cell culture apparatus according to the embodiment.

FIG. 4 is a diagram explaining the operations of the culture container in the cell culture process.

FIG. 5 is a flowchart of a cell peeling process performed by the cell culture apparatus according to the embodiment.

FIG. 6 is a diagram explaining the operations of the culture container in the cell peeling process.

FIG. 7 is a diagram explaining the operations of the cell culture apparatus in the cell peeling process.

FIG. 8 is a diagram explaining the operations of the cell culture apparatus in the cell peeling process.

FIG. 9 is a flowchart of a cell concentration process performed by the cell culture apparatus according to the embodiment.

FIG. 10 is a diagram explaining the operations of the culture container in the cell concentration process.

### DETAILED DESCRIPTION

In general, according to one embodiment, a cell culture apparatus includes a culture container, a motor, an ejecting pipe, and a discharging pipe. The culture container has a transverse hollow tubular shape, has an opening formed in the center of a bottom surface of the culture container, and has an openable and closable lid portion formed on a side surface of the culture container. The motor rotates the culture container around a rotation axis perpendicular to the bottom surface. The ejecting pipe is inserted from the opening into the culture container. The discharging pipe is connected to the lid portion. An inner wall surface of the culture container is a surface for culturing cells, and has a shape of a single spiral when viewed from the direction of the rotation axis. The ejecting pipe ejects a liquid onto the inner wall surface. The discharging pipe discharges the ejected liquid from the lid portion to the outside of the culture container.

Hereinafter, a cell culture apparatus and a cell culture method according to an embodiment will be described with reference to the accompanying drawings. In the embodiment (s) described below, elements assigned with the same reference numerals perform the same operations, and redundant descriptions will be omitted as appropriate.

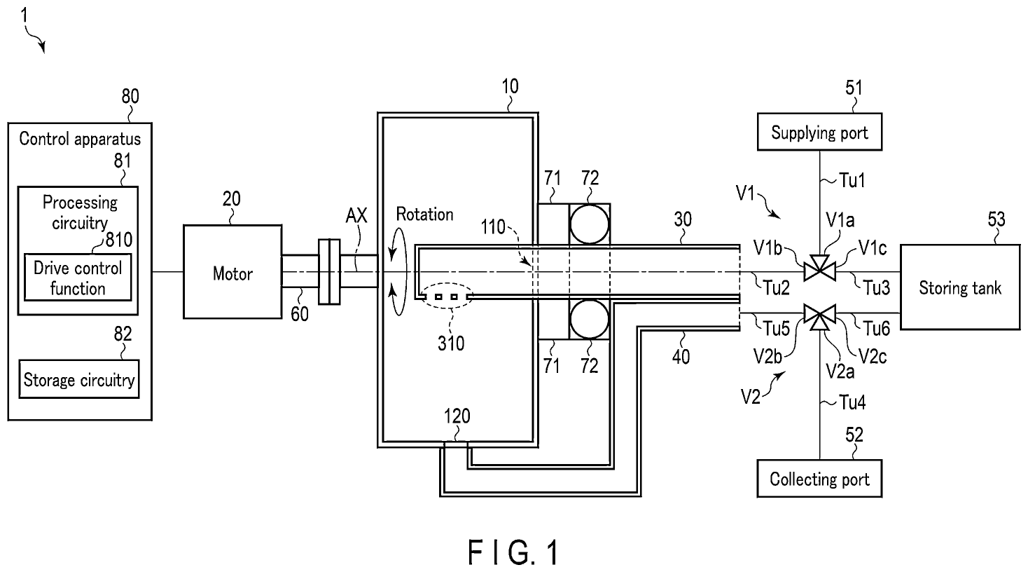

FIG. 1 is a schematic diagram showing an example of a configuration of a cell culture apparatus 1 according to an embodiment. The cell culture apparatus 1 is a closed-system cell culture apparatus and automatically cultures cells while keeping the inside of the cell culture apparatus 1 sterile. The cell culture apparatus 1 includes a culture container 10, a motor 20, an ejecting pipe 30, a discharging pipe 40, a supplying port 51, a collecting port 52, a storing tank 53, a connection portion 60, a spacer 71, a bearing 72, and a control apparatus 80. For convenience of explanation, a cross-section of the cell culture apparatus 1 viewed in the frontal direction is shown to explain a partial structure (i.e., the culture container 10, the ejecting pipe 30, the discharging pipe 40, the spacer 71, and the bearing 72) of the cell culture apparatus 1.

The culture container 10 is a container for culturing cells. The culture container 10 is made of plastic, polycarbonate, aluminum, etc. The culture container 10 has a transverse hollow tubular shape. An opening 110 is formed in the center of one of the bottom surfaces of the culture container 10. An openable and closable lid portion 120 is formed on a side surface of the culture container 10. An inner wall surface of the culture container 10 is a surface for culturing cells. The lid portion 120 may be formed on the bottom surface of the culture container 10 (e.g., the periphery of the bottom surface on which the opening 110 is formed).

In the embodiment, the lid portion 120 of the culture container 10 is connected to a lid portion driver (not shown). The lid portion driver opens or closes the lid portion 120 according to the control executed by the control apparatus 80. Any method (e.g., one-side opening, double-side opening, slide opening) may be applied to the opening and closing of the lid portion 120.

The motor 20 is configured to drive the rotation of the culture container 10. The motor 20 is connected to the center of the other of the bottom surfaces of the culture container 10 via the connection portion 60. Thus, the motor 20 rotates the culture container 10 around a rotation axis AX perpendicular to the bottom surface of the culture container 10. The motor 20 rotates the culture container 10 at a predetermined speed according to the control executed by the control apparatus 80. The motor 20 is an example of a rotation driver.

The ejecting pipe 30 is a pipe for ejecting a liquid into the inside of the culture container 10. The ejecting pipe 30 is made of plastic, polycarbonate, aluminum, etc. The ejecting pipe 30 is inserted from the opening 110 formed on the bottom surface of the culture container 10 into the culture container 10. In this state, the ejecting pipe 30 ejects a liquid from an ejecting port 310 formed at a distal end onto the inner wall surface of the culture container 10. An angle of ejecting a liquid from the ejecting pipe 30 is freely adjusted. A central axis of the ejecting pipe 30 coincides with the rotation axis AX of the culture container 10. The ejecting pipe 30 may be formed of two portions, a portion that is inserted from the opening 110 into the culture container 10 and a portion that ejects a liquid onto the inner wall surface of the culture container 10. The ejecting pipe 30 may be a nozzle. The ejecting pipe 30 is an example of an ejector.

In the embodiment, the annular spacer 71 and the bearing 72 are attached to the ejecting pipe 30. The spacer 71 is arranged to be in contact with an outer wall surface of the bottom surface of the culture container 10. The bearing 72 is arranged to be in contact with the spacer 71. Thus, the culture container 10 rotates around the rotation axis AX along an outer circumferential side surface of the ejecting pipe 30, preventing the rotation of the ejecting pipe 30. Accordingly, the ejecting pipe 30 can eject a liquid onto the entire inner wall surface of the culture container 10 while the culture container 10 is rotating.

The discharging pipe 40 is a pipe for discharging a liquid from the inside of the culture container 10. The discharging pipe 40 is made of plastic, polycarbonate, aluminum, etc. The discharging pipe 40 is connected to the lid portion 120 when the rotation of the culture container 10 is stopped. Thus, the discharging pipe 40 discharges, from the lid portion 120 to the outside of the culture container 10, the liquid ejected by the ejecting pipe 30. The discharging pipe 40 is an example of a discharger.

The lid portion 120 may be positioned in a lower part of the culture container 10 when the rotation of the culture container 10 is stopped. The liquid ejected by the ejecting pipe 30 is accumulated in the lower part of the culture container 10 due to gravitation. Therefore, the discharging pipe 40 can efficiently discharge the liquid accumulated in the lower part of the culture container 10 from the lid portion 120 positioned in the lower part of the culture container 10.

The supplying port 51 is a port for supplying a liquid. The supplying port 51 is connected to a container that contains a cell suspension or a reagent. The supplying port 51 is connected to a branch portion V1 by a tube Tu1.

The branch portion V1 is formed of valves V1a, V1b, and V1c. The valve V1a is connected to the supplying port 51 by the tube Tu1. The valve V1b is connected to the ejecting pipe 30 by a tube Tu2. The valve V1c is connected to the storing tank 53 by a tube Tu3. Each valve of the branch portion V1 has a valve driver (not shown). Each valve driver opens or closes each valve according to the control executed by the control apparatus 80.

The collecting port 52 is a port for collecting a liquid. The collecting port 52 is connected to a collecting container or a waste tank. The collecting port 52 is connected to a branch portion V2 by a tube Tu4.

The branch portion V2 is formed of valves V2a, V2b, and V2c. The valve V2a is connected to the collecting port 52 by the tube Tu4. The valve V2b is connected to the discharging pipe 40 by a tube Tu5. The valve V2c is connected to the storing tank 53 by a tube Tu6. Each valve of the branch portion V2 has a valve driver (not shown). Each valve driver opens or closes each valve according to the control executed by the control apparatus 80.

The storing tank 53 is a tank for storing a liquid. The storing tank 53 is connected to the branch portion V1 by the tube Tu3 and connected to the branch portion V2 by the tube Tu6. The storing tank 53 has a pump (not shown). The pump delivers the liquid stored in the storing tank 53 to the ejecting tube 30 through the tubes Tu2 and Tu3 with a predetermined pressure according to the control executed by the control apparatus 80. Further, the pump suctions the liquid discharged by the discharging pipe 40 into the storing tank 53 through the tubes Tu5 and Tu6 with a predetermined pressure according to the control executed by the control apparatus 80.

The control apparatus 80 is an apparatus for controlling the operations of the cell culture apparatus 1. The control apparatus 80 has processing circuitry 81 and storage circuitry 82. The control apparatus 80 may further have an input interface, a communication interface, a display, etc.

The processing circuitry 81 is circuitry for controlling the operations of the cell culture apparatus 1. The processing circuitry 81 includes at least one processor. The term “processor” means circuitry such as a CPU (central processing unit), a GPU (graphics processing unit), an ASIC (application specific integrated circuit), a programmable logic device (for example, an SPLD (simple programmable logic device), a CPLD (complex programmable logic device), or an FPGA (field programmable gate array)), etc. If the processor is a CPU, the CPU implements each function by reading and executing the programs stored in the storage circuitry 82. If the processor is an ASIC, each function is directly incorporated as logic circuitry into the circuitry of the ASIC. The processor may be constituted in the form of single circuitry or in the form of multiple independent sets of circuitry combined. In the embodiment, the processing circuitry 81 implements a drive control function 810.

The drive control function 810 is a function of controlling the operations of the cell culture apparatus 1. Firstly, the drive control function 810 controls the opening and the closing of the lid portion 120 of the culture container 10 by controlling the lid portion driver. Secondly, the drive control function 810 rotates the culture container 10 at a predetermined speed by controlling the motor 20. Thirdly, the drive control function 810 controls the opening and the closing of each valve by controlling each valve driver of the branch portions V1 and V2. Fourthly, the drive control function 810 delivers the liquid stored in the storing tank 53 to the ejecting pipe 30 or suctions the liquid discharged by the discharging pipe 40 into the storing tank 53 by controlling the pump.

The storage circuitry 82 stores various kinds of data. For example, the storage circuitry 82 stores information relating to cell culturing (e.g., the type of cells, the type of medium, the time that has elapsed after the initiation of a cell culture, the time that has elapsed after the previous replacement of the medium), control information of the various drivers (e.g., the number of rotations), various kinds of programs, etc. For example, the storage circuitry 82 is realized by a semiconductor memory device (e.g., a random access memory (RAM), a flash memory), a hard disk, an optical disk, or the like.

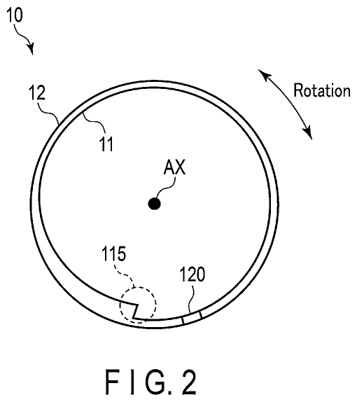

FIG. 2 is a schematic diagram showing an example of a configuration of the culture container 10 of the cell culture apparatus 1 according to the embodiment. FIG. 2 shows a cross-section of the culture container 10 viewed from the direction of the rotation axis AX in FIG. 1.

An inner wall surface 11 of the culture container 10 has a shape of a single spiral when viewed from the direction of the rotation axis AX. A protrusion 115 that has a hook shape (L-shape) and includes both ends of the spiral is formed on the inner wall surface 11. On the other hand, an outer wall surface 12 of the culture container 10 has a shape of a precise circle when viewed from the direction of the rotation axis AX. That is, the thickness of the side surface of the culture container 10 gradually increases in the area from the end of the spiral farther from the rotation axis AX to the end of the spiral closer to the rotation axis AX.

The lid portion 120 of the culture container 10 is formed on a portion of the side surface farther from the rotation axis AX in the vicinity of the protrusion 115. That is, the lid portion 120 is formed on a portion of the side surface where the culture container 10 is thinner. The lid portion 120 may be formed in a position spaced from the protrusion 115 to a certain degree so that cells can be accumulated on a portion of the side surface between the protrusion 115 and the lid portion 120.

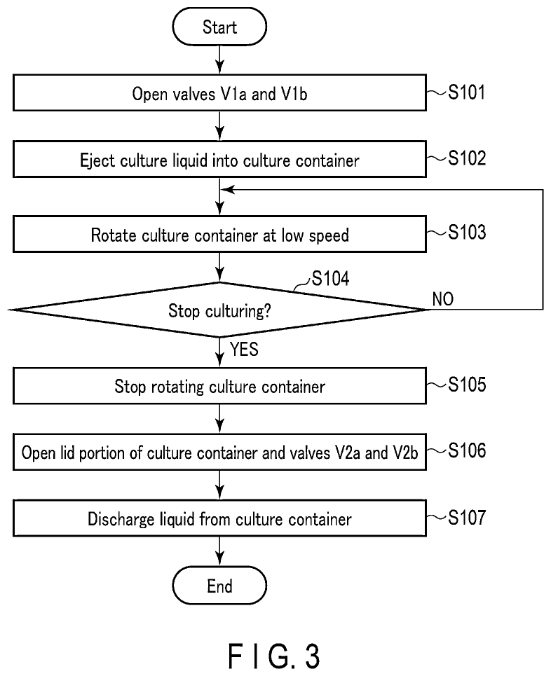

FIG. 3 is a flowchart of a cell culture process performed by the cell culture apparatus 1 according to the embodiment. In the process shown in FIG. 3, the control apparatus 80 of the cell culture apparatus 1 performs each step of the flowchart by implementing the drive control function 810.

As a condition for starting the process, a container containing a cell culture liquid is connected to the supplying port 51. A waste tank for collecting a liquid is connected to the collecting port 52. The lid portion 120 of the culture container 10 and all the valves are closed. Adhesive cells are seeded on the inner wall surface of the culture container 10.

First, the drive control function 810 opens the valves V1a and V1b using each valve driver (step S101). The drive control function 810 can thereby supply the culture liquid in the container connected to the supplying port 51 to the culture container 10 through the tubes Tu1 and Tu2.

Next, the drive control function 810 ejects a culture liquid onto the inner wall surface of the culture container 10 using the pump and the ejecting pipe 30 (step S102) and rotates the culture container 10 at a low speed using the motor 20 (step S103). Steps S102 and S103 may be performed simultaneously.

The drive control function 810 then determines whether or not to stop a culture (step S104). For example, the drive control function 810 determines to stop a culture if a culture period set in advance elapses. Alternatively, the drive control function 810 determines to stop a culture if it receives an instruction to stop a culture from an operator through an input interface. If the drive control function 810 determines to stop a culture (step S104—YES), the process proceeds to step S105. If the drive control function 810 determines not to stop a culture (step S104—NO), the process returns to step S103.

Subsequently, the drive control function 810 stops rotating the culture container 10 using the motor 20 (step S105) and opens the lid portion 120 of the culture container 10 and the valves V2a and V2b using the lid portion driver and the respective valve drivers (step S106). The drive control function 810 can thereby collect the liquid accumulated inside the culture container 10 into the waste tank connected to the collecting port 52 through the discharging pipe 40 and the tubes Tu4 and Tu5.

Lastly, the drive control function 810 discharges the liquid from the lid portion 120 of the culture container 10 to the waste tank using the pump and the discharging pipe 40 (step S107). After step S107, the drive control function 810 ends the series of steps.

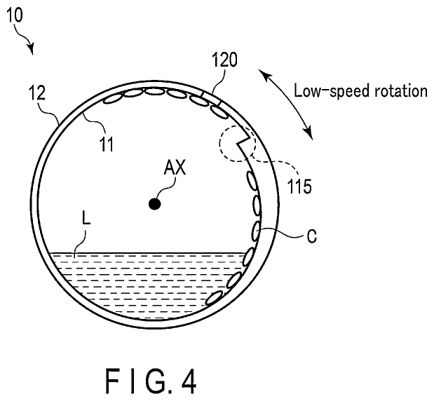

FIG. 4 is a diagram explaining the operations of the culture container 10 in the cell culture process. As in FIG. 2, FIG. 4 shows a cross-section of the culture container 10 viewed from the direction of the rotation axis AX in FIG. 1. In particular, FIG. 4 explains the operations of the culture container 10 in step S103 shown in FIG. 3.

Cells C are seeded on the inner wall surface 11 of the culture container 10. Specifically, the cells C are seeded in a semi-perimeter of the inner wall surface 11 having the protrusion 115 in the center. A liquid L is accumulated in the lower part of the culture container 10. With the culture container 10 rotating around the rotation axis AX at a low speed in this state, the liquid L can come into full contact with all of the cells C on the inner wall surface 11.

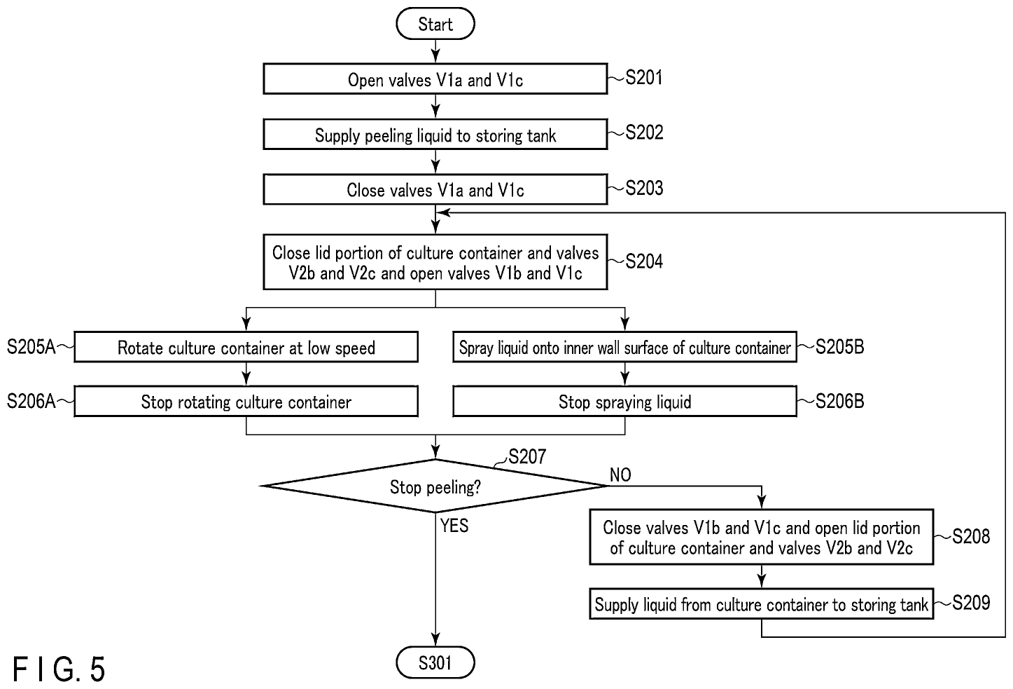

FIG. 5 is a flowchart of a cell peeling process performed by the cell culture apparatus 1 according to the embodiment. In the process shown in FIG. 5, the control apparatus 80 of the cell culture apparatus 1 performs each step of the flowchart by implementing the drive control function 810, as in the process shown in FIG. 3.

As a condition for starting the process, a container containing a cell peeling liquid is connected to the supplying port 51. A waste tank for collecting a liquid is connected to the collecting port 52. The lid portion 120 of the culture container 10 and all the valves are closed. Adhesive cells are seeded on the inner wall surface of the culture container 10.

First, the drive control function 810 opens the valves V1a and V1c using each valve driver (step S201). The drive control function 810 can thereby supply the peeling liquid in the container connected to the supplying port 51 to the storing tank 53 through the tubes Tu1 and Tu3.

Next, the drive control function 810 supplies the peeling liquid to the storing tank 53 using the pump (step S202) and closes the valves V1a and V1c using each valve driver (step S203). The peeling liquid is thereby stored in the storing tank 53.

Subsequently, the drive control function 810 closes the lid portion 120 of the culture container 10 and the valves V2b and V2c using the lid portion driver and the respective valve drivers and opens the valves V1b and V1c (step S204). The drive control function 810 can thereby supply the liquid stored in the storing tank 53 to the culture container 10 through the tubes Tu2 and Tu3.

Subsequently, the drive control function 810 sprays the liquid onto the inner wall surface of the culture container 10 using the pump and the ejecting pipe 30 (step S205B) while rotating the culture container 10 at a low speed using the motor 20 (step S205A). At this time, the drive control function 810 ejects the liquid with a pressure higher than the pressure with which the cell culture is performed. The drive control function 810 may spray the liquid until all the liquid stored in the storing tank 53 is delivered.

Subsequently, the drive control function 810 stops rotating the culture container 10 using the motor 20 (step S206A) and also stops spraying the liquid using the pump and the ejecting pipe 30 (step S206B).

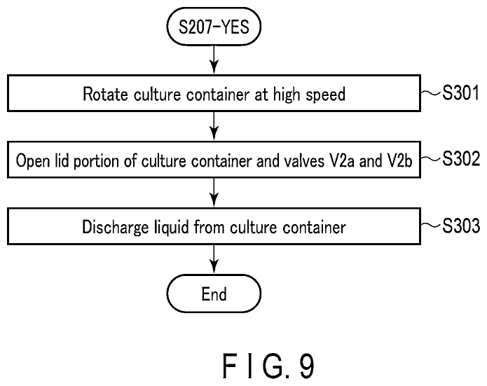

The drive control function 810 then determines whether or not to stop peeling (step S207). For example, the drive control function 810 determines to stop peeling if the liquid is sprayed a preset number of times. Alternatively, the drive control function 810 determines to stop peeling if it receives an instruction to stop peeling from an operator through an input interface. If the drive control function 810 determines to stop peeling (step S207—YES), the process proceeds to step S301 (see FIG. 9). If the drive control function 810 determines not to stop peeling (step S207—NO), the process proceeds to step S208.

Subsequently, the drive control function 810 closes the valves V1b and V1c and opens the lid portion 120 of the culture container 10 and the valves V2b and V2c using the lid portion driver and the respective valve drivers (step S208). The drive control function 810 can thereby supply the liquid accumulated inside the culture container 10 to the storing tank 53 through the tubes Tu5 and Tu6.

Subsequently, the drive control function 810 supplies the liquid from the lid portion 120 of the culture container 10 to the storing tank 53 using the pump and the discharging pipe 40 (step S209). After step S209, the process returns to step S204. That is, steps S204 through S209 are repeatedly performed any number of times.

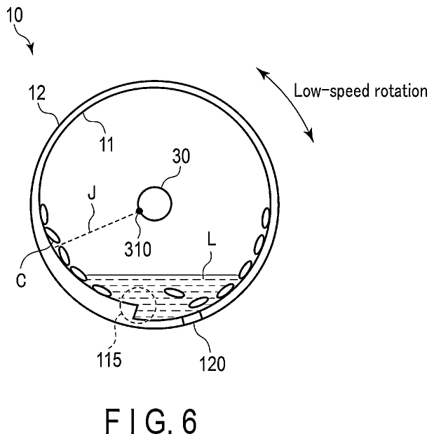

FIG. 6 is a diagram explaining the operations of the culture container 10 in the cell peeling process. As in FIGS. 2 and 4, FIG. 6 shows a cross-section of the culture container 10 viewed from the direction of the rotation axis AX in FIG. 1. In particular, FIG. 6 explains the operations of the culture container 10 in steps S205A and S205B (shown in FIG. 5) performed for the first and the subsequent time.

The cells C are cultured on the inner wall surface 11 of the culture container 10. The ejecting pipe 30 ejects the liquid L onto the cells C cultured on the inner wall surface 11 with a predetermined pressure while the culture container 10 is being rotated. Thus, a jet J of the liquid L sprayed from the ejecting port 310 of the ejecting pipe 30 peels the cells C from the inner wall surface 11. The peeled cells C are suspended in the liquid L accumulated in the lower part of the culture container 10.

Since the liquid L is accumulated in the lower part of the culture container 10, it is better for the ejecting pipe 30 to eject the liquid L obliquely downward rather than to eject the liquid L toward the right under the ejecting pipe 30. The jet J of the liquid L thereby reaches the inner wall surface 11 without letting its pressure be decreased due to the liquid L accumulated in the lower part of the culture container 10. Thus, the ejecting pipe 30 can more efficiently peel the cells C from the inner wall surface 11.

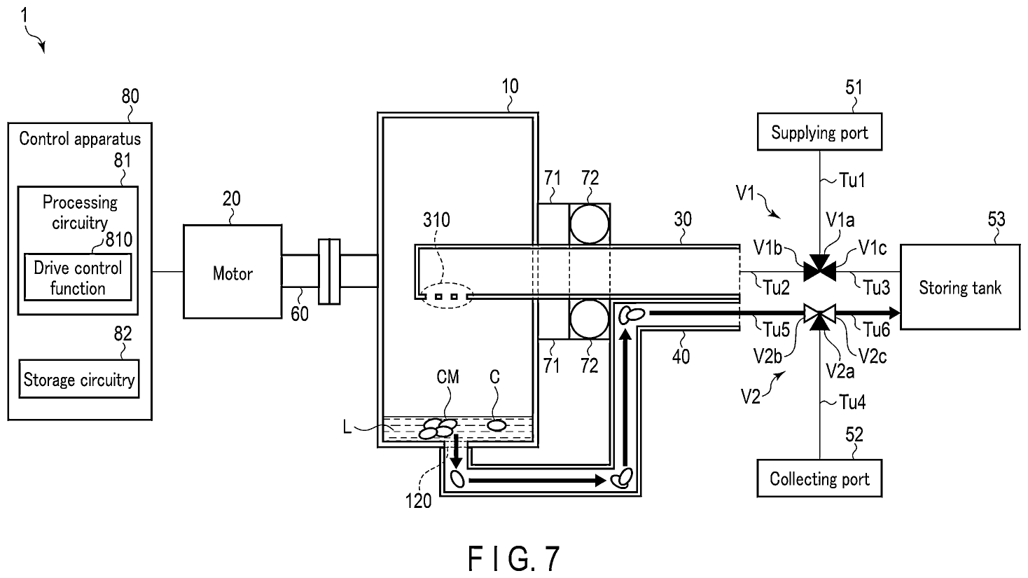

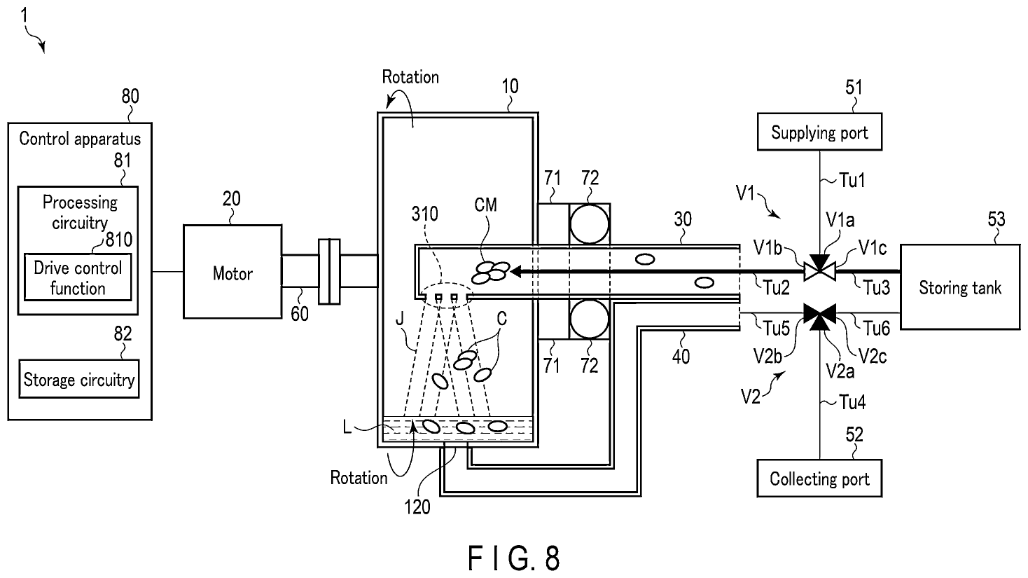

FIGS. 7 and 8 are diagrams explaining the operations of the cell culture apparatus 1 in the cell peeling process. In FIGS. 7 and 8, a cross-section of the cell culture apparatus 1 viewed in the frontal direction is shown to explain a partial structure (i.e., the culture container 10, the ejecting pipe 30, the discharging pipe 40, the spacer 71, and the bearing 72) of the cell culture apparatus 1, as in FIG. 1. Also, in FIGS. 7 and 8, the valves in an open state are indicated by a white triangle, and the valves in a closed state are indicated by a black triangle. In particular, FIG. 7 explains the operation of the cell culture apparatus 1 in step S209 (shown in FIG. 5) performed for the first and the subsequent time. FIG. 8 explains the operations of the cell culture apparatus 1 in steps S205A and S205B (shown in FIG. 5) performed for the second and the subsequent time.

As shown in FIG. 7, the cells C are peeled from the inner wall surface of the culture container 10 and suspended in the liquid L. The cells C suspended in the liquid L are supplied from the lid portion 120 of the culture container 10 to the storing tank 53 through the discharging pipe 40 and the tubes Tu5 and Tu6. Some of the cells C may be peeled from the inner wall surface of the culture container 10 in a state of being adhered to each other and form a cell mass CM. The cell mass CM suspended in the liquid L is supplied to the storing tank 53 through the same paths as those described above.

As shown in FIG. 8, the cells C and the cell mass CM supplied to the storing tank 53 are supplied to the ejecting pipe 30 through the tubes Tu2 and Tu3. The ejecting pipe 30 separates the cells C from each other by ejecting the supplied cells C and cell mass CM from the ejecting port 310 with a predetermined pressure. The cells C forming the cell mass CM are thereby separated into discrete cells C.

That is, the cell culture apparatus 1 circulates the liquid L and the cells C through a flow path in the ejecting pipe 30 and a flow path in the discharging pipe 40. Since the cell culture apparatus 1 repeatedly ejects the cells C collected from the discharging pipe 40 from the ejecting pipe 30, it can efficiently separate the cells C.

FIG. 9 is a flowchart of a cell concentration process performed by the cell culture apparatus 1 according to the embodiment. In the process shown in FIG. 9, the control apparatus 80 of the cell culture apparatus 1 performs each step of the flowchart by implementing the drive control function 810, as in the processes shown in FIGS. 3 and 5. The process shown in the flowchart is a series of steps that follow step S207—YES in FIG. 5.

First, the drive control function 810 rotates the culture container 10 at a high speed using the motor 20 (step S301). At this time, the drive control function 810 rotates the culture container 10 an increased number of times as compared to the number of rotations at the time of the cell culture and the number of rotations at the time of the cell peeling.

Next, the drive control function 810 opens the lid portion 120 of the culture container 10 and the valves V2a and V2b using the lid portion driver and the respective valve drivers (step S302). The drive control function 810 can thereby collect the liquid accumulated inside the culture container 10 into the waste tank connected to the collecting port 52 through the discharging pipe 40 and the tubes Tu4 and Tu5.

Lastly, the drive control function 810 discharges the liquid from the lid portion 120 of the culture container 10 to the waste tank using the pump and the discharging pipe 40 (step S303). After step S303, the drive control function 810 ends the series of steps.

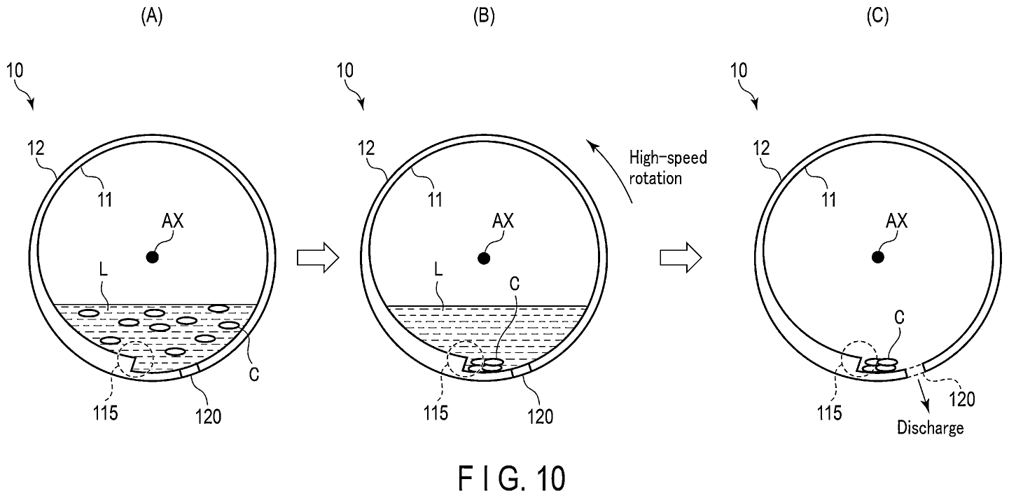

FIG. 10 is a diagram explaining the operations of the culture container 10 in the cell concentration process. As in FIGS. 2, 4 and 6, FIG. 10 shows a cross-section of the culture container 10 viewed from the direction of the rotation axis AX in FIG. 1. In particular, FIG. 10 (A) shows the outside of the culture container 10 before step S301 shown in FIG. 9 is performed. FIG. 10 (B) shows the outside of the culture container 10 after step S301 shown in FIG. 9 is performed. FIG. 10 (C) shows the outside of the culture container 10 after step S303 shown in FIG. 9 is performed.

As shown in FIG. 10 (A), the liquid L in which the cells C are suspended is accumulated in the lower part of the culture container 10. All the cells C are peeled from the inner wall surface 11 of the culture container 10.

As shown in FIG. 10 (B), the inner wall surface 11 of the culture container 10 has a shape of a single spiral when viewed from the direction of the rotation axis AX. The culture container 10 concentrates the cells C suspended in the liquid L in a position between the protrusion 115 and the lid portion 120 by rotating at a high speed in a direction of the spiral toward the rotation axis AX (in a counterclockwise direction in FIG. 10 (B)).

Specifically, the cells C suspended in the liquid L are accumulated over the inner wall surface 11 by a centrifugal force generated by the high-speed rotation of the culture container 10. In tandem with the rotation of the culture container 10, the protrusion 115 accumulates, on the surface of the protrusion 115, the cells accumulated on the inner wall surface 11. In this manner, the protrusion 115 can efficiently concentrate and collect the cells C suspended in the liquid L.

As shown in FIG. 10 (C), the liquid L accumulated in the culture container 10 is discharged from the lid portion 120 to the outside of the culture container 10. The lid portion 120 is formed in a position spaced from the protrusion 115 to a certain degree. Thus, the culture container 10 can efficiently discharge the liquid L without discharging the cells C accumulated near the protrusion 115.

Above are descriptions of the cell culture apparatus 1 according to the embodiment. According to the embodiment, the cell culture apparatus 1 can efficiently perform, in a single culture container, a process necessary for cell culturing in a closed-system. Thus, the cell culture apparatus 1 can contribute to mechanization of a process relating to cell culturing, mass-production of cells through culturing, etc.

Especially in the concentration process, the cell culture apparatus 1 concentrates the cell suspension using centrifugation, not a filter. Thus, the cell culture apparatus 1 can prevent the loss of cells caused by the use of a filter. Furthermore, the cell culture apparatus 1 collects the concentrated cells using the protrusion 115 formed on the inner wall surface 11 in tandem with the high-speed rotation of the culture container 10. The cell culture apparatus 1 removes the supernatant from the lid portion 120 positioned near the protrusion 115. That is, since the cell culture apparatus 1 need not transfer the cell suspension in the culture container 10 to a different container to perform configuration, the cell culture apparatus 1 can be realized with a simple structure.

In addition, the cell culture apparatus 1 can efficiently carry out culturing various cells including adhesive cells (e.g., iPS cells) and floating cells (e.g., hematopoietic stem cells).

The shape, etc., of the culture container 10 of the cell culture apparatus 1 is not limited to those shown in the embodiment. According to another embodiment, the culture container 10 may be a cylindrical body having a bottom surface forming an elliptical shape. Furthermore, the inner wall surface 11 of the culture container 10 may have a shape of a precise circle instead of a spiral shape. In this case, a protrusion having any shape may be formed in at least one portion of the inner wall surface 11 of the culture container 10.

According to at least one embodiment described above, the process relating to cell culturing can be supported.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A cell culture apparatus comprising:
a culture container having a transverse hollow tubular shape, having an opening formed in a center of a bottom surface of the culture container, and having an openable and closable lid portion formed on a side surface of the culture container;
a motor configured to rotate the culture container around a rotation axis perpendicular to the bottom surface;
an ejecting pipe inserted from the opening into the culture container; and
a discharging pipe connected to the lid portion,
wherein
an inner wall surface of the culture container is a surface for culturing cells and has a shape of a single spiral when viewed from a direction of the rotation axis,
the ejecting pipe ejects a liquid onto the inner wall surface, and
the discharging pipe discharges the ejected liquid from the lid portion to an outside of the culture container.

2. The cell culture apparatus according to claim 1, wherein the lid portion is formed on a portion of the side surface farther from the rotation axis, the lid portion is formed near a protrusion, and the protrusion is formed on the inner wall surface so as to include both ends of the spiral.

3. The cell culture apparatus according to claim 2, wherein the culture container concentrates the cells suspended in the liquid in a position between the protrusion and the lid portion by rotating in a direction of the spiral toward the rotation axis.

4. The cell culture apparatus according to claim 1, wherein the ejecting pipe ejects the liquid onto the cells cultured on the inner wall surface during rotation of the culture container, thereby peeling the cells from the inner wall surface.

5. The cell culture apparatus according to claim 1, wherein
the discharging pipe supplies the cells suspended in the liquid to the ejecting pipe, and
the ejecting pipe ejects the supplied cells with a predetermined pressure, thereby separating the cells from each other.

6. The cell culture apparatus according to claim 1, wherein the discharging pipe is connected to the lid portion when the rotation of the culture container is stopped.

7. A cell culture method performed by a cell culture apparatus,
the cell culture apparatus comprising:
a culture container having a transverse hollow tubular shape, having an opening formed in a center of a bottom surface of the culture container, and having an openable and closable lid portion formed on a side surface of the culture container;
a motor configured to rotate the culture container around a rotation axis perpendicular to the bottom surface;
an ejecting pipe inserted from the opening into the culture container; and
a discharging pipe connected to the lid portion,
an inner wall surface of the culture container being a surface for culturing cells and has a shape of a single spiral when viewed from a direction of the rotation axis,
the method comprising:
a first step in which the ejecting pipe ejects a liquid onto the inner wall surface; and
a second step in which the discharging pipe discharges the ejected liquid from the lid portion to an outside of the culture container.

8. The cell culture method according to claim 7, wherein
the lid portion is formed on a portion of the side surface farther from the rotation axis, the lid portion is formed near a protrusion, and the protrusion is formed on the inner wall surface so as to include both ends of the spiral, and
the method further comprises, between the first step and the second step, a third step in which the culture container concentrates the cells suspended in the liquid in a position between the protrusion and the lid portion by rotating in a direction of the spiral toward the rotation axis.

9. The cell culture method according to claim 7, wherein, in the first step, the ejecting pipe ejects the liquid onto the cells cultured on the inner wall surface during rotation of the culture container, thereby peeling the cells from the inner wall surface.

10. The cell culture method according to claim 7, further comprising, after the second step:
a third step in which the discharging pipe supplies the cells suspended in the liquid to the ejecting pipe; and
a fourth step in which the ejecting pipe ejects the supplied cells with a predetermined pressure, thereby separating the cells from each other.
